(19) United States
(12) Patent Application Publication
(10) Pub. No.: US 2013/0237781 A1
(43) Pub. Date: Sep. 12, 2013

(54) Transcutaneous Device

(71) Applicant: Steffen Gyrn, Ringsted (DK)

(72) Inventor: Steffen Gyrn, Ringsted (DK)

(21) Appl. No.: 13/823,631

(22) PCT Filed: Sep. 28, 2011
(86) PCT No.: PCT/EP2011/066912

§ 371 Date: May 20, 2013

Related U.S. Application Data
(60) Provisional application No. 61/387,170, filed on Sep. 28, 2010.

(30) Foreign Application Priority Data
Sep. 28, 2010 (EP) 10180817.8

(51) Int. Cl. A61M 5/158; A61B 5/157

(52) U.S. Cl. CPC A61M 5/158; A61B 5/157
USPC 600/309; 604/174

(57) ABSTRACT

The present application relates to a transcutaneous device for use in delivery and/or sensor devices or the like. The transcutaneous device is of a type which can be used as a component in different types of delivery and/or sensor devices and which is applied after a base part has been applied to the patient's skin. The application relates to a transcutaneous device to be secured to a base part which base part during use is secured to a patient's skin, the transcutaneous device comprises a housing and a subcutaneous part secured unreleasably to the housing, said transcutaneous device is provided with attachment means attaching the transcutaneous device to the base part. The attachment means comprises more than one inwardly or outwardly protruding parts sequentially arranged in direction of insertion along an outer surface of the body of the transcutaneous device. The direction of insertion is coinciding with the direction in which the insertion needle points during insertion.

The present invention relates to a transcutaneous device for use in delivery and/or sensor devices or the like. The transcutaneous device is of a type which can be used as a component in different types of delivery and/or sensor devices and which is applied after a base part has been applied to the patient's skin.

### PRIOR ART

Often delivery devices for intermittent or continuous administration of a therapeutical substance, such as insulin, are in form of a two-part device. Such a traditional delivery device comprises a base part having a cannula for subcutaneous insertion into a patient and means for fastening the base part to the patient's skin. The base part has means for closing and/or opening of fluid access to the base part. The means for closing and/or opening of fluid access can e.g. be means for receiving a connector cannula extending from a connector part and for bringing the connector cannula into fluid communication with the cannula of the base part. Often, the connector part is in fluid communication with a drug delivery device, e.g. an insulin pump.

WO 2008/014791 shows a cannula device for mounting in a base part, wherein the base part during use is secured to a patient's skin surface. The cannula device comprises a housing and at least one membrane, where the membrane and the housing together define at least one cavity. The cannula device further comprises a cannula secured unreleasably to the housing, wherein the cannula is in fluid communication with the at least one cavity. The cannula device is provided with means for attaching the device to the base part.

When the cannula device is inserted into the base plate, normally using an inserter, the cannula device has to get firmly attached to the base plate in order to be released from the inserter after insertion. According to WO 2008/014791, the cannula device is attached to the base part by means of 1) mechanical hooks on the cannula device, which get caught against a skin facing surface of the base part, or 2) a skin facing adhesive surface of the cannula device, which touches an oppositely positioned surface of the base part.

No matter how the explicit embodiment of a cannula device is caught and attached to the base part, it is necessary to obtain a certain contact between the cannula device and the base part in order for the cannula device to be adequately secured to the base plate. If the cannula device is not adequately secured to the base plate, it will not be released from the inserter when the insertion needle is removed from the inserted cannula device, e.g. by automatic retraction into an automatic inserter. It is therefore necessary that the cannula device is inserted into a certain depth in order to be released from the inserter.

### DESCRIPTION

The object of the present invention is to provide a transcutaneous device such as a sensor device, a cannula device or the like, having a larger tolerance for positioning during insertion. Normally, the transcutaneous device is relatively small and cannot be handled without an inserter. The transcutaneous device is to be secured to a base part, such as e.g. a delivery device, which base part during use is secured to a patient's skin. The transcutaneous device comprises a body and a subcutaneous part secured unreleasably to the body. The subcutaneous part can e.g. be a cannula. Said transcutaneous device is provided with attachment means attaching the transcutaneous device to the base part. The attachment means comprises an area extending in the entire length of or part of the length of the body of the transcutaneous device which area is provided with either a continuous layer able to provide a friction larger than the force attaching the transcutaneous device to the inserting device, i.e. providing a frictional resistance between the base part and the transcutaneous device larger than the power attaching the transcutaneous device to the insertion needle device, or comprises more than one inwardly or outwardly protruding part sequentially arranged in direction of insertion along an outer surface of the body of the transcutaneous device which layer or parts provide more than one position to attach the transcutaneous device to the base part.

After the transcutaneous device has been inserted by the aid of the inserter, the inserter is retracted from the transcutaneous device. Which injection depth the subcutaneous part reaches can depend on e.g. the inserter used for the insertion, the size of the transcutaneous device in relation to the size of the base part, the patient's skin and other external factors.

After insertion of the transcutaneous device, the inserter is retracted. When using the prior art solution, described in e.g. WO 2008/014791, it is necessary that the transcutaneous device is inserted to a projected injection depth in order for the transcutaneous device to be firmly locked in position in the base part. If the transcutaneous device does not reach this projected injection depth, the transcutaneous device cannot be released from the inserter and it will consequently be pulled out of the patient's skin again causing discomfort to the patient.

With the transcutaneous device according to the invention, it is ensured that the transcutaneous device is firmly secured in the base part even if there are unpredictable variations in the projected injection depth caused by differences in the physical dimensions of the parts and/or the assembly process.

By the transcutaneous device according to the invention, it is further ensured that upon retraction of the inserter, the distance the transcutaneous device can move upward in relation to the base part is minimized. This ensures a tight fitting of the transcutaneous device in the base part.

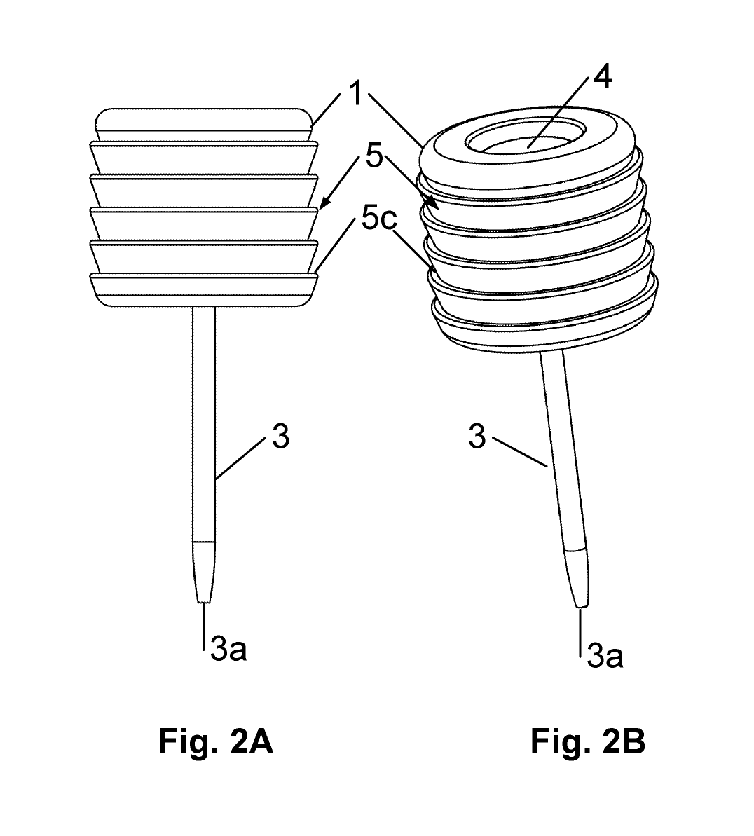

According to one or more embodiments of a transcutaneous device according to the invention, the attachment means comprises more than one circumferentially positioned inwardly or outwardly protruding part. In other words, the attachment means can be positioned as a band along the surface of the body of the transcutaneous device. Thus, if the body is cylindrical, the circumferential surface is circular and the attachment means are circular bands positioned on the outer surface of the body, as e.g. in the embodiments shown in FIGS. 2, 3 and 6. Accordingly, if the body is rectangular, it has a quadrangular or square circumferential surface and the attachment means are quadrangular or square bands.

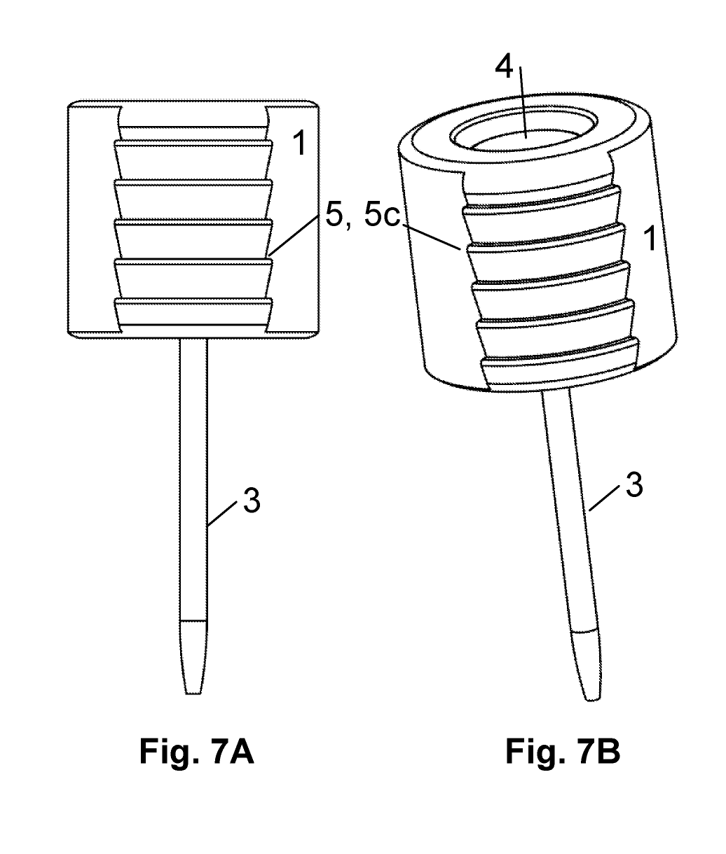

According to one or more embodiments of a transcutaneous device according to the invention, the attachment means comprises inwardly or outwardly protruding parts having a length of e.g. less than a fourth of the circumference of the body at the level where the part is positioned. Such an embodiment is shown in FIG. 7. By length is meant the length of the protruding part extending in a direction substantially parallel to the surface of the patient's skin, thus a direction substantially perpendicular to the direction of insertion.

According to one or more embodiments of a transcutaneous device according to the invention, at least a part of the outer surface of the body of the transcutaneous device is plane and can slide and guide the transcutaneous device along the inner surface of an inserter. Such an embodiment is shown in FIG. 7.

According to one or more embodiments of a transcutaneous device according to the invention, the attachment means comprises at least three inwardly or outwardly protruding parts sequentially arranged in the direction of insertion along an outer surface of the body of the transcutaneous device. Often, the attachment means will comprise at least four inwardly/outwardly protruding parts. The upper limit for how many inwardly/outwardly protruding parts, which can be positioned along the surface of the body of the transcutaneous device, is determined by the dimensions of the attachment means and the dimensions of the body of the transcutaneous device.

According to one or more embodiments of a transcutaneous device according to the invention, the subcutaneous part is a cannula being in fluid contact with an internal cavity of the body.

According to one or more embodiments of a transcutaneous device according to the invention, the subcutaneous part is a sensor being in electric or fluid contact with a part of the body and able to register one or more components or conditions.

According to one or more embodiments of a transcutaneous device according to the invention, the attachment means is cooperating with corresponding means on the base part. According to this embodiment of the invention, the corresponding means on the base part might be flexible, thereby enabling the protruding parts of the attachment means to pass partly or fully at least in one direction. The corresponding means might be flexible either as a consequence of being made in a soft and flexible material such as Thermo Plastic Elastomers (TPE) or as a consequence of being to some extent bendable.

According to one or more embodiments of a transcutaneous device according to the invention, the body comprises a proximal surface through which the subcutaneous part extends, wherein the proximal surface comprises a recess surrounding the subcutaneous part, thereby creating an open space around the subcutaneous part. This allows for the subcutaneous part to form a bulge in the recess, thereby preventing the cannula to move upwards again after assembly into the housing.

The cannula device will be described in further detail with reference to the figures, wherein:

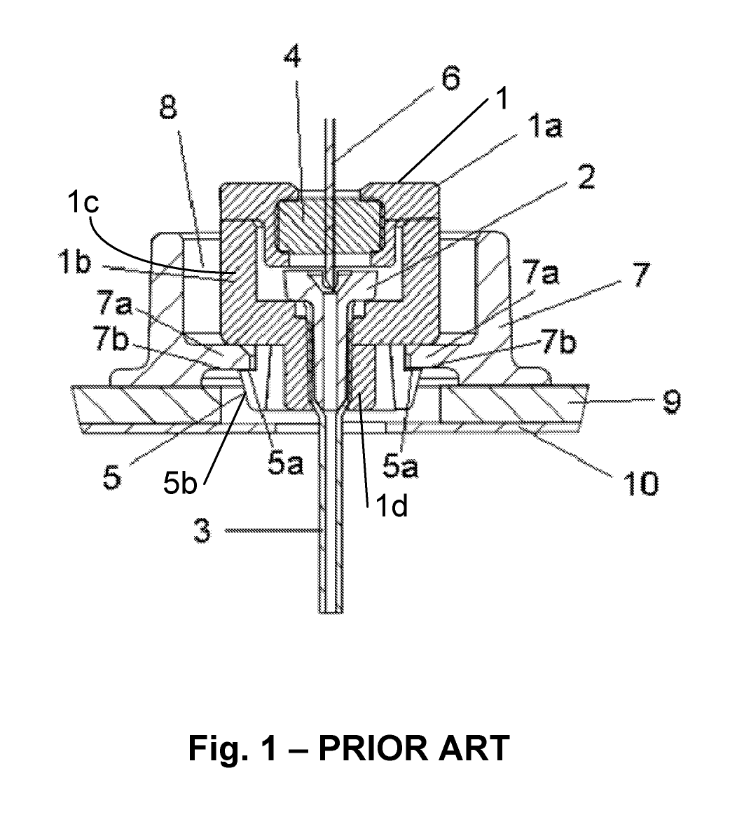

FIG. 1 is a cut-through view of an embodiment of a cannula device of the prior art placed in a receiving portion of a delivery device;

FIGS. 2A-B show an embodiment of a transcutaneous device according to the invention, wherein the transcutaneous devices is a cannula part;

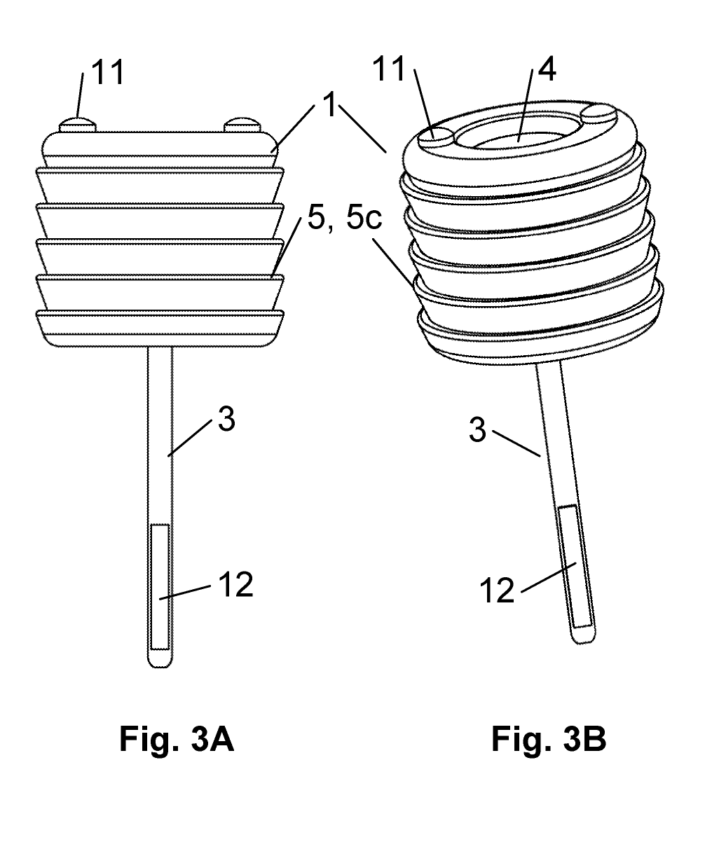

FIGS. 3A-B show an embodiment of a transcutaneous device according to the invention, wherein the transcutaneous device is a sensor part;

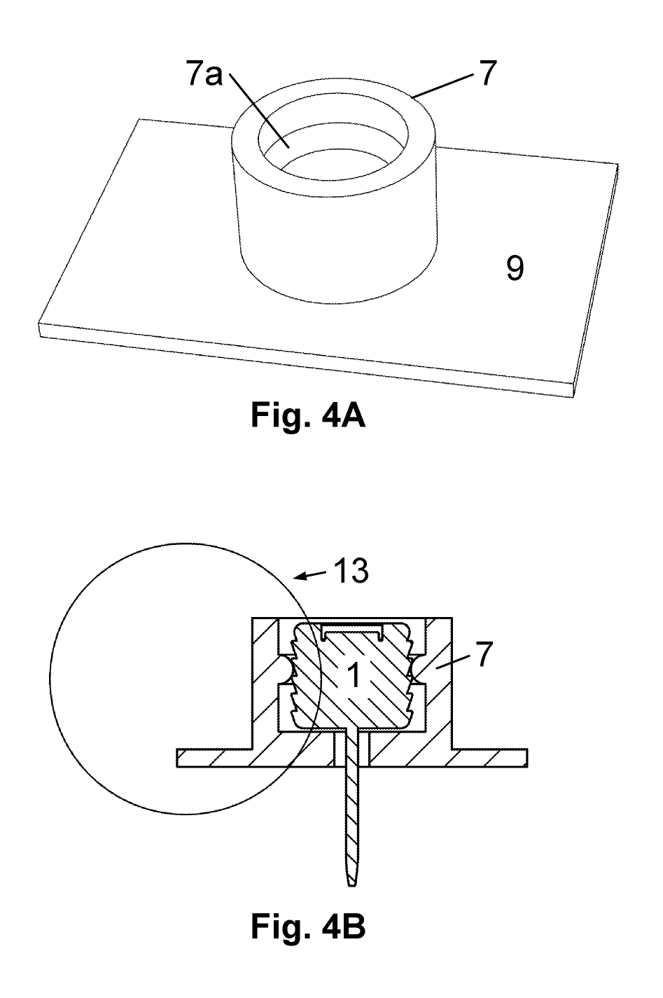

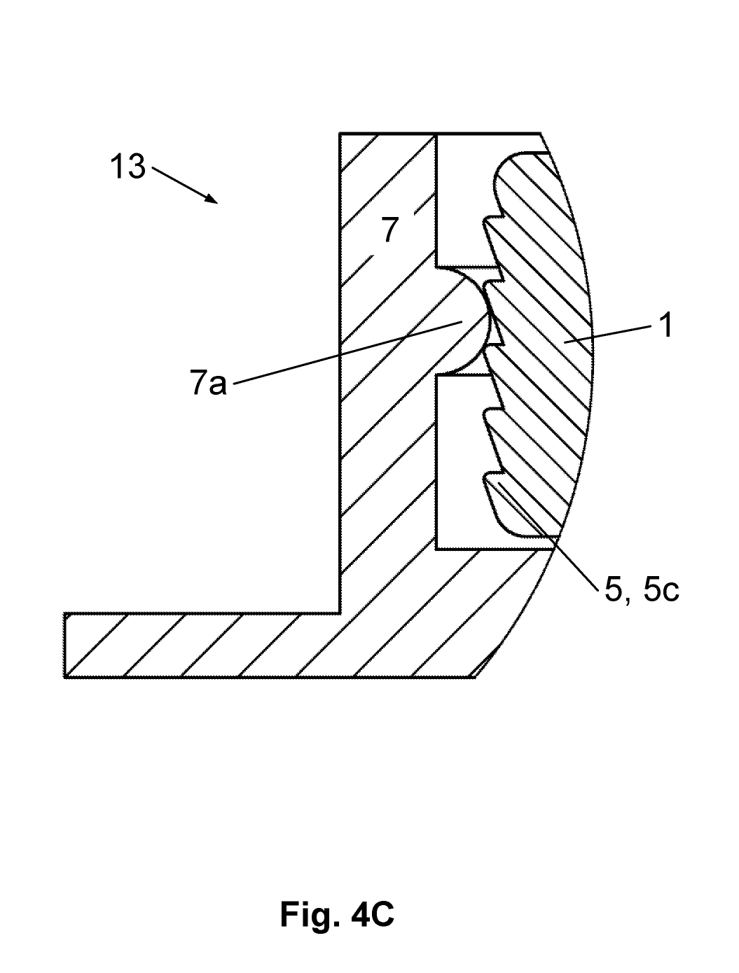

FIGS. 4A-C show a first embodiment of a receiving portion of a base part (FIG. 4A), a cut-through view of a cannula device placed in the receiving portion of the base part (FIG. 4B), and an enlargement showing how the attachment means of the transcutaneous device cooperates with corresponding means placed on the inwardly facing surface of the receiving portion (FIG. 4C);

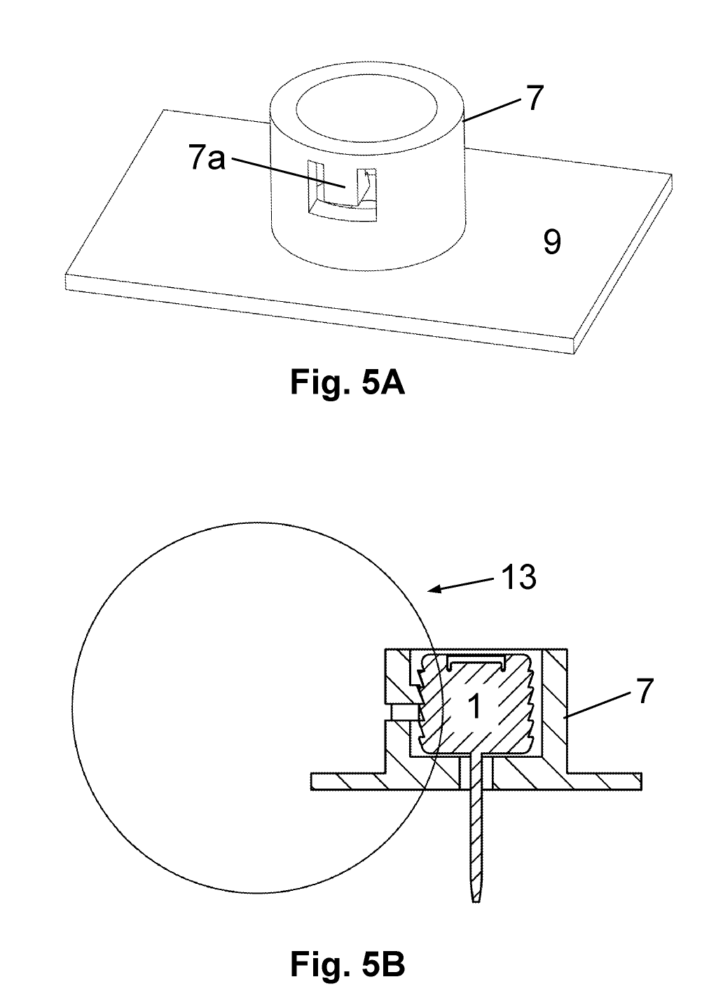

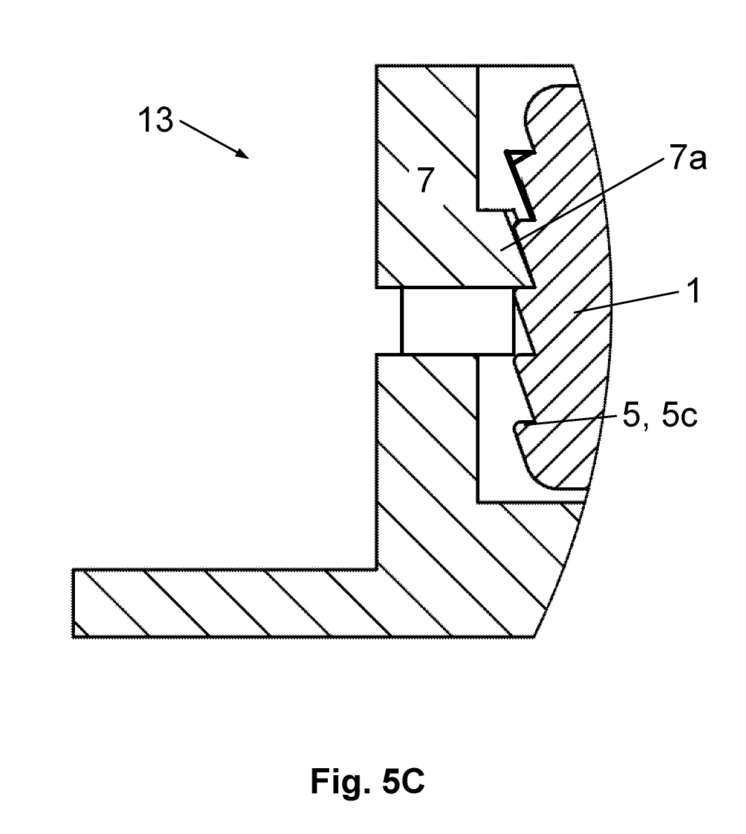

FIGS. 5A-C show a second embodiment of a receiving portion of a base part (FIG. 5A), a cut-through view of a cannula device placed in the receiving portion of the base part (FIG. 5B), and an enlargement showing how the attachment means of the transcutaneous device cooperates with corresponding means placed on the inwardly facing surface of the receiving portion (FIG. 5C);

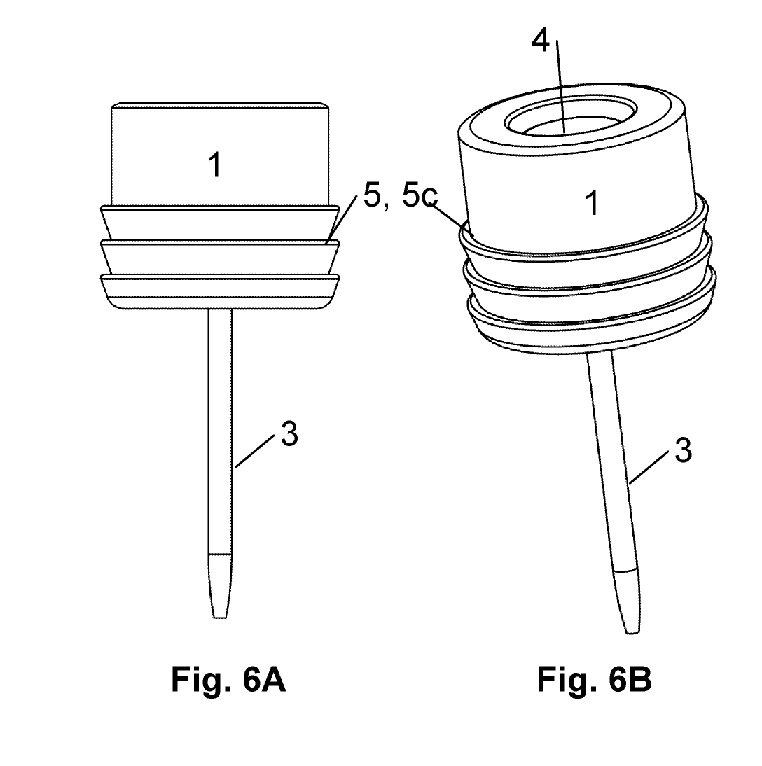

FIGS. 6A-B show an embodiment of a body of a transcutaneous device where only the lower or proximal end of the body is provided with attachment means;

FIGS. 7A-B show an embodiment of a body of a transcutaneous device where approximately 25% of the circumference is provided with attachment means;

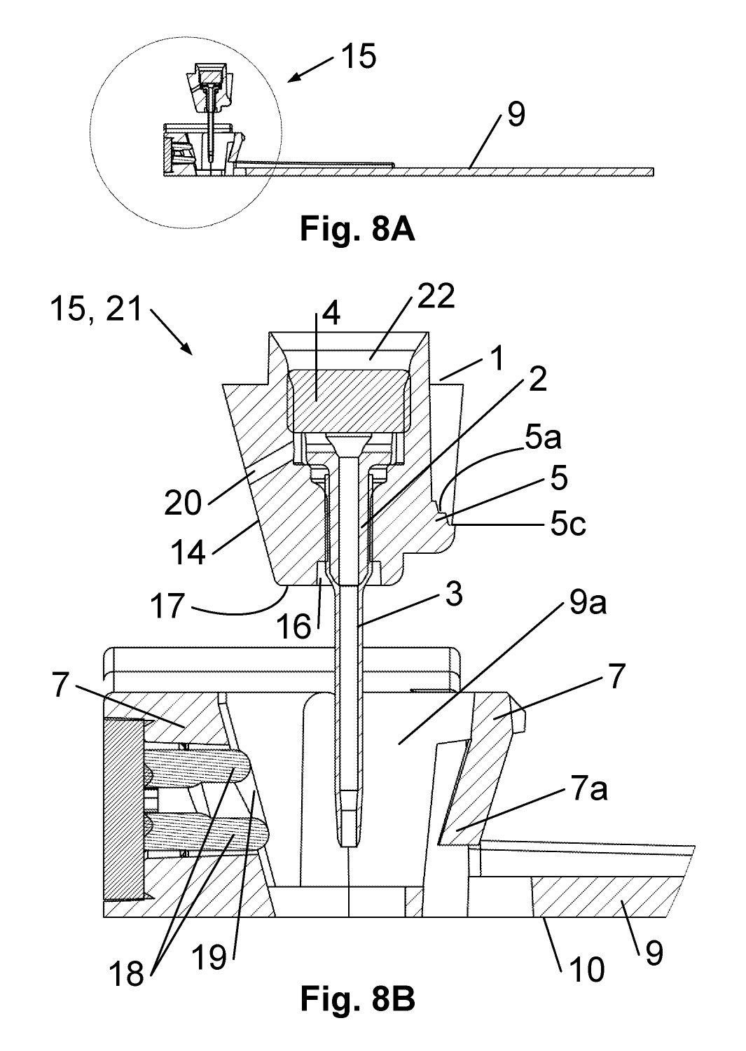

FIGS. 8A-B show a cut-through view of an embodiment of a transcutaneous device before being placed in a receiving portion of a delivery device; and

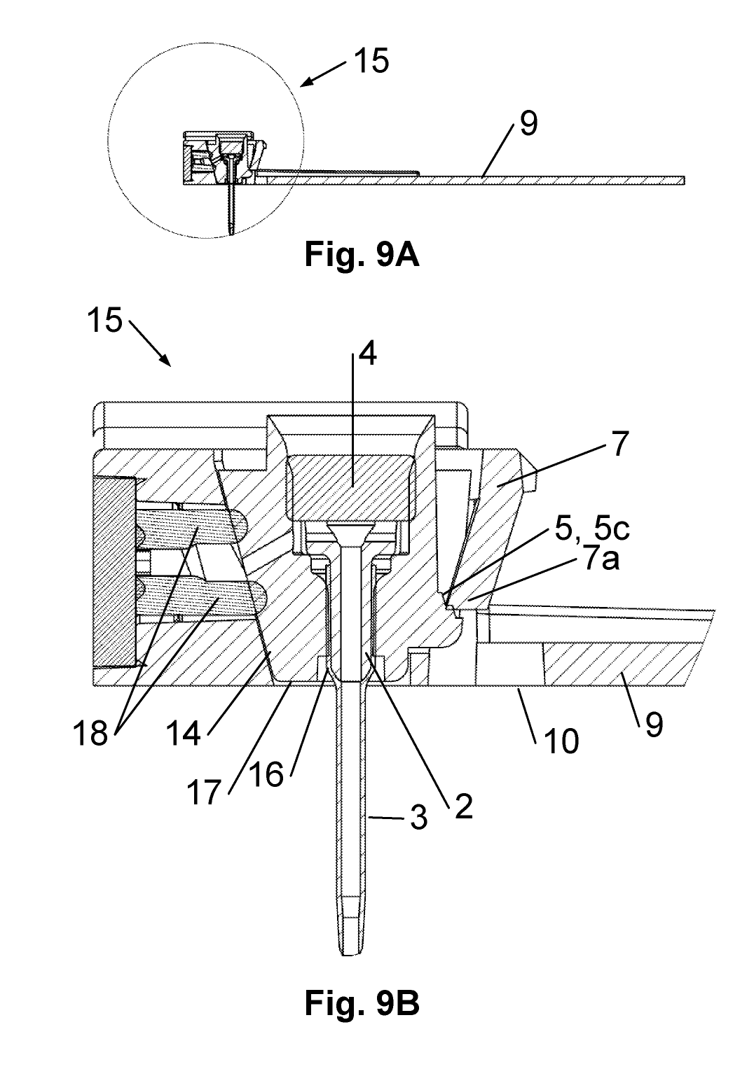

FIGS. 9A-B show a cut-through view of the embodiment of a transcutaneous device in FIGS. 8A-B placed in the receiving portion of the delivery device.

### DETAILED DESCRIPTION OF THE INVENTION

FIG. 1 shows a known cannula device comprising a two-part housing 1 with a top part 1a and a bottom part 1b. In the top part 1a of the housing there is a wall 4 in the form of e.g. a membrane. The two-part housing and the wall define an inner cavity adapted to receive a piercing member 6 extending from e.g. a connector or a syringe. The two-part housing 1a, 1b is normally made of a relatively hard moulded plastic material.

The bottom part 1b of the housing can be constructed of two cylindrical parts; an upper cylindrical part 1c and a lower cylindrical part 1d, the lower cylindrical part 1d having a smaller diameter than the upper cylindrical part 1c. The inner surface of the upper cylindrical part 1c forms the wall of the inner cavity. The outer surface of the upper cylindrical part 1c is smooth and without protrusions. The inner surface of the lower cylindrical part 1d forms an opening which supports a cannula 3. The outer surface of the lower cylindrical part comprises means 5 for attaching the cannula device unreleasably to a base part 9. The prior art comprises one set of attachment means 5 placed in the same horizontal level. The attachment means 5 consequently only allows for insertion of the cannula device in one position at one vertical level. By horizontal is meant the direction substantially perpendicular to the direction of insertion also being substantially parallel with the surface of the patient's skin. By vertical is meant the direction substantially parallel with the direction of insertion and also being substantially perpendicular to the surface of the patient's skin.

The cannula 3 is made of a soft inert material. In this embodiment the cannula 3 is attached to the housing part 1b by pushing a fastening part/bushing 2 made of a more rigid material than the cannula 3 into the opening of the lower cylindrical part 1d after positioning the cannula 3 in the opening. As the fastening part/bushing 2 is pushed into the opening, the cannula 3 will be squeezed against the walls of the opening and this pressure will keep the cannula 3 in a correct position.

The means 5 for attaching the cannula device unreleasably to the base part 9 is in this prior art embodiment constructed as multiple hooks, which can flex around the position where they are attached to the bottom part 1b of the housing. In this prior art embodiment, the hooks are attached in a flexible manner, using e.g. a flexible material, thus allowing the hooks to be pushed inwards when the hooks 5 pass a transition of reduced clearance. Each hook is provided with an upper surface 5a parallel to a contact surface 7b of a receiving portion 7 of the base part 9. Each hook is also provided with an inclined surface 5b which during insertion of the cannula device is in contact with a protruding part 7a of the receiving portion 7.

When the cannula device is pushed down into the receiving portion 7, the hooks are pushed inwards against the lower cylindrical part 1d of the housing. As the hooks in this position are biased, the hooks will return to their original position when the inclined surfaces 5b of the hooks have fully passed the protruding part 7a of the receiving portion 7. When the hooks return to their original position, the upper surfaces 5a of the hooks will be in touch with the contact surfaces 7b of the receiving portion 7 and the cannula device will be locked in this position as neither the cannula device nor the receiving portion 7 are provided with means to push the hooks inward against the lower cylindrical part 1d of the housing.

Alternatively, the upper side of the protruding parts 7a or the lower side of the cylindrical part 1c can be provided with an adhesive which then works as unreleasably attaching means when the cannula device is pushed into position in the receiving portion 7. This construction of the attachment means necessitates that the upper surface 5a of the attachment means 5 during insertion of the subcutaneous part 3 has to be brought to an insertion level where the hooks are allowed to pivot to the outward position such that the upper surface 5a can get into contact with the lower surface 7b of the receiving portion of the base part 9.

If the subcutaneous part does not reach an adequate injection depth, the subcutaneous part will be retracted together with the insertion needle when the insertion needle is retracted. This means that the prior art embodiment shown in FIG. 1 is strongly susceptible to even small variations in injection depth resulting from physical dimensions of the parts and/or assembly processes.

FIG. 1 shows the cannula device positioned in the receiving portion 7 provided with essentially vertically positioned walls covering the side section of the cannula device and a bottom part formed by the protruding parts 7a on which the cannula device rests when locked in the receiving portion 7. The space 8 around the cannula device, which has the form of a cylindrical room between the essentially vertical walls and the side section of the cannula device, creates a guiding mean for an inserter. The cannula device is in this prior art embodiment normally fully covered by a lower cylindrical part of the inserter, and when the user wants to inject the cannula device, the cylindrical lower part of the inserter is placed in the space 8 formed in the receiving portion 7. The cannula device can then be pushed in position by a plunger moving the cannula device forward inside the cylindrical lower part of the inserter. The receiving portion 7 is as a consequence attached unreleasably to the base part 9, which is again fastened to the skin of a patient e.g. with a mounting pad 10.

When describing the embodiments of the invention shown in FIGS. 2-9 reference numbers of parts corresponding to parts described in connection with the prior art of FIG. 1 are used.

FIGS. 2A-B and 3A-B show embodiments of two examples of transcutaneous devices according to the invention; a cannula device (FIGS. 2A-B) and a sensor device (FIGS. 3A-B).

FIGS. 2A-B show a transcutaneous device seen in a front view (FIG. 2A) and in a perspective view (FIG. 2B). The transcutaneous device in FIGS. 2A-B comprises a body 1 corresponding to the housing 1 in FIG. 1, which might be produced as a two-part structure like the prior art, and a subcutaneous part 3 secured unreleasably to the body 1.

According to the embodiment shown in FIGS. 2A-B, the subcutaneous part 3 is a cannula through which medication, such as insulin, can flow from an inner not shown cavity of the body 1 to an open end 3a of the cannula 3. The inner cavity is protected from the surroundings by a membrane 4 through which an insertion needle can pass. The insertion needle is used if the cannula is a soft cannula unable to penetrate the patient's skin by itself.

The transcutaneous device is provided with attachment means 5 for attaching the transcutaneous device to the base part 9 when the transcutaneous device is subcutaneously inserted bringing the attachment means 5 into contact with corresponding means 7a on the base part. The interaction between the attachment means 5 and the corresponding means 7a of the base part 9 will result in the transcutaneous part being attached unreleasably to the base part 9 after having been inserted.

According to the shown embodiment in FIGS. 2A-B, the attachment means 5 comprises five protruding parts 5c placed sequentially in the direction of insertion along an outer surface of the body 1 of the transcutaneous device. As the protruding parts 5c of the attachment means 5 are distributed equally all the way around the body 1 of the transcutaneous device and as the body is circular, the protruding parts 5c of the attachment means 5 form circular bands, each band being placed at its own horizontal level.

FIGS. 3A-B show a transcutaneous device seen in a front view (FIG. 3A) and in a perspective view (FIG. 3B). According to the embodiment shown in FIGS. 3A-B, the subcutaneous part 3 is a sensor provided with a contact area 12 which is able to register the amount of at least one component or at least one condition in the patient's subcutaneous layer. Signals registered by the contact area 12 are transported to the contact areas 11 at the top surface of the transcutaneous device. From the contact areas 11, the signals can be read and e.g. used to control administration of medication given to the patient. If the sensor needs to be inserted by a separate insertion needle, a membrane 4 can be used to protect the open passage needed for the insertion needle.

The transcutaneous device is provided with attachment means 5 which will cause the transcutaneous device to be attached to the base part 9 when the transcutaneous device is subcutaneously inserted and the attachment means 5 is brought into contact with corresponding means 7a on the base part. The interaction between the attachment means 5 and the corresponding means 7a of the base part 9 will result in the transcutaneous part being attached unreleasably to the base part 9 after having been inserted.

According to the shown embodiment in FIGS. 3A-B, the attachment means 5 comprises five protruding parts 5c placed sequentially in the direction of insertion along an outer surface of the body 1 of the transcutaneous device. As the protruding parts 5c of the attachment means 5 are distributed equally all the way around the body 1 of the transcutaneous device and as the body is circular, the protruding parts 5c of the attachment means 5 form circular bands, each band being placed at its own horizontal level.

According to another embodiment, the attachment means 5 comprises a continuous surface e.g. constituted of a rough material or another surface material providing increased friction extending in the direction of insertion. The corresponding means 7a on the base part 9 comprises a protruding part allowing the continuous surface constituting the attachment means 5 to pass in sliding contact with the corresponding means 7a when the transcutaneous device moves in the direction of insertion, but does not allow sliding contact when the transcutaneous device is moved in the direction opposite the insertion direction.

Allowing sliding contact in one direction and blocking it in the opposite direction can be done in several ways. Normally it is done by combining a rough area on the transcutaneous device extending in a direction of insertion (e.g. in the whole length or a part of the length of the body of the transcutaneous device) with a corresponding means 7a comprising a protruding part made of e.g. thermoplastic material or another rubber like elastic material which can provide increased friction when sliding against the surface of the attachment means 5. Basically, the contact between the continuous surface of the attachment means 5 and the corresponding means 7a should provide a friction between the base part and the transcutaneous device larger than the power attaching the transcutaneous device to the insertion needle device.

The coefficient of friction is an empirical measurement—it has to be measured experimentally, and cannot be found through calculations. Rougher surfaces tend to have higher effective values. Both static and kinetic coefficients of friction depend on the respective surfaces in contact.

FIG. 4A shows a receiving portion 7 of a base part 9 with corresponding means 7a placed on the inwardly facing surface of the receiving portion 7. FIG. 4B shows a cut-through view of a transcutaneous device 1, e.g. a cannula device or a sensor device, placed in the receiving portion 7 of the base part 9, and FIG. 4C shows the enlargement 13 marked with a circle in FIG. 4B showing how the attachment means 5 of the transcutaneous device 1 cooperates with corresponding means 7a placed on the inwardly facing surface of the receiving portion 7 of the base part 9.

The attachment means 5 comprises a series of protruding parts, having a triangular cross-section, placed on the outer surface of the body 1. The triangular parts have an upwardly facing plane surface almost parallel to the upper surface of the base part 9 and an inclined downwardly facing surface which during insertion will slide along the corresponding means 7a.

The corresponding means 7a comprises an elastic mass arranged as a circular band protruding from the inwardly facing surface of the receiving portion 7 in such a way that the attachment means 5 displaces the elastic mass when respective protruding parts 5c of the attachment means 5 pass the band 7a. When the respective protruding parts 5c of the attachment means 5 have passed the elastic mass, the elastic mass recover its form and the corresponding means 7a prevents that the just passed protruding parts 5c of the attachment means 5 move upwards.

When the inserter has reached its maximum injection depth, the insertion needle attached to the inserter will be retracted from the transcutaneous part, while the transcutaneous device will not be allowed to move upwards with the inserter as at least one of the protruding parts 5c of the attachment means 5 has passed the elastic mass 7a. Upon retraction of the inserter the maximum distance the transcutaneous device can move upward is the distance between two succeeding protruding parts 5c of attachment means 5. Thus, a tight fitting of the transcutaneous device in the delivery device is ensured, independent of the actual injection depth.

FIGS. 5A-C show an embodiment similar to the embodiment of FIGS. 4A-C, wherein FIG. 5A shows a receiving portion 7 of a base part 9 with corresponding means 7a, FIG. 5B shows a cut-through view of a transcutaneous device 1, e.g. a cannula device or a sensor device, placed in the receiving portion 7 of the base part 9, and FIG. 5C shows the enlargement 13 marked with a circle in FIG. 5B.

In the embodiment of FIGS. 5A-C, the elasticity of the corresponding means 7a is not obtained by using an elastic material but by using a part of the relatively rigid material which is used to make the receiving portion 7. The elasticity is provided by cutting out three sides of a section of the wall of the receiving portion in such a way that the cut out section can pivot relative to the remaining upper side. Like the embodiment shown in FIGS. 4A-C, the inclined surface of the attachment means can slide along the inward surface of the pivotal portion 7a while an upward movement of the body of the transcutaneous part will be prevented as soon as the pivotal portion 7a gets in contact with an upwardly facing plane surface of a protruding part of 5c attachment means 5.

FIGS. 6A-B show an embodiment of a transcutaneous device according to the invention (front view in FIG. 6A and perspective view in FIG. 6B), wherein the attachment means 5 of the transcutaneous device only comprises three serially positioned protruding parts 5c positioned at the lower or proximal end of the body 1 of the transcutaneous device. This embodiment allows for a good sliding contact between the upper and distal part of the body 1 of the transcutaneous device and the inserter.

FIGS. 7A-B show an embodiment of a transcutaneous device according to the invention (front view in FIG. 7A and perspective view in FIG. 7B), wherein the attachment means 5 does not cover the entire circumference of the body 1 of the transcutaneous device. This embodiment instead has attachment means 5 comprising five protruding parts 5c sequentially arranged in the vertical direction at the outer surface of the body of the transcutaneous device. The length of all of the attachment means is around 25% of the complete circumference. By length is meant the length of the protruding part extending in a direction substantially parallel to the surface of the patient's skin, thus a direction substantially perpendicular to the direction of insertion.

According to another embodiment, a similar series of protruding parts 5c could be placed on the opposite surface area of body 1 of the transcutaneous device thereby providing a balanced configuration on the transcutaneous device after insertion.

The distance between the succeeding protruding parts 5c need not be the same; this is valid for all the embodiments of the invention.

FIGS. 8A-B show a cut-through view of an embodiment of a transcutaneous device 21, e.g. a cannula device or a senor device, just before the transcutaneous device is inserted in a receiving portion of a delivery device 9, and FIGS. 9A-B the transcutaneous device 21 inserted in the receiving portion of the delivery device 9. FIG. 8B and 9B are enhancement views of the area 15 marked with the circle in FIG. 8A and 9A, respectively.

The delivery device 9 comprises an outlet opening 19 for delivery of fluid, e.g. insulin, from the delivery device 9 to the transcutaneous device 21 and there from to the patient after insertion of the transcutaneous device 21. The outlet opening 19 is provided with an elastic sealing 18 ensuring a fluid tight connection between the outlet opening 19 and the transcutaneous device 21.

The transcutaneous device 21 comprises a body 1 with a wall 4 in the form of e.g. a membrane, wherein the body 1 and the wall 4 define an inner cavity adapted to receive a piercing member extending from e.g. a connector or a syringe. The body 1 is normally made of a relatively hard moulded plastic material.

The body 1 is provided with a cannula 3 and a flat inclining surface 14 provided with an opening 20 corresponding to the outlet opening 19 resulting in fluid contact between the two corresponding openings 19, 20, when they are positioned opposite each other, thus allowing unrestricted flow.

The flat inclining surface 14 is inclined in order to reduce the force required to insert the transcutaneous device 21 in the delivery device 9. When the transcutaneous device 21 is inserted, it will be press-fitted into the receiving portion 9a of the delivery device 9 and the elastic sealing 18 will provide a completely fluid tight gasket around the corresponding openings 19 and 20. In FIG. 9A-B, the elastic sealing 18 is shown as protruding into the body 1, which is due to a mistake in the drawing program. In reality, the elastic sealing 18 does not protrude into the body 1, but is pressed against the body 1 in a press-fit configuration.

In order to improve the press-fitting and thereby the fluid tight connection between the transcutaneous device 21 and the outlet 19 of the fluid path, the receiving portion 9a of the delivery device 9 can be provided with a decreasing cross-section in the direction of insertion. The body 1 of the transcutaneous device 21 will have a corresponding decreasing cross-section.

The inclining surface 14 of the transcutaneous device 21 does not need to be flat; it can have any desired shape as long as it is possible to create a corresponding surface on the receiving portion 9a of the delivery device 9. Also, the opening 20 of the inclining surface 14 can be an inlet or an outlet depending on the purpose of the transcutaneous device 21.

The top opening 22 of the body 1 is covered with a self closing membrane 4 through which an inserter needle extends during inserting of a transcutaneous device 21 with the cannula 3 being a soft cannula. However, the top opening 22 can also be used as an additional port for injecting medication or nutrients other than the primary medication e.g. insulin which the patient receives via the opening 20.

The transcutaneous device 21 is provided with attachment means 5 in the form of a staircase comprising protruding parts 5c. The attachment means 5 is positioned on part of the body 1 opposite the flat inclining surface 14. The attachment means 5 ensures that the transcutaneous device 21 after insertion is secured in the delivery device 9 by interactions with the corresponding means 7a on the delivery device 9.

The corresponding means 7a is flexible, thus allowing it to be pushed outward when the attachment means 5 passes during insertion of the transcutaneous device 21. The individual protruding parts 5c of the staircase in the attachment means 5 each comprise an upwardly facing surface 5a, which can be locked by the downward surface of the corresponding means 7a of the delivery device 9.

This ensures that it will not be possible to detach the transcutaneous device 21 from the delivery device 9. In the shown embodiment, the attachment means 5 comprises three stair steps, but the attachment means 5 could also have two or more than three steps depending on e.g. the requirements to tolerance and the size of the transcutaneous device 21 and the delivery device 9.

In FIGS. 9A-B, the transcutaneous device 21 is locked in a middle position by the middle stair step. Which stair step that will be locked by the corresponding means 9a depends on the depth at which the transcutaneous device 21 is inserted. Thus, the multiple outwards protruding stair steps of the attachment means 5 eliminate the tolerance limit set by only having one hook as in the prior art shown in FIG. 1.

When the inserter used for insertion of the transcutaneous device 21 has reached its maximum injection depth, the insertion needle along with the inserter will be retracted from the transcutaneous device 21. Upon retraction of the inserter, the maximum distance the transcutaneous device 21 can move upward is the distance between two succeeding stair steps of attachment means 5. Variations in the injection depths can be prompt by e.g. minor variations in the size of the body 1 in relation to the receiving portion 9a of the delivery device 9, the inserter used for the insertion, the patient's skin or other external factors. However, by having multiple steps in the attachment means 5, a tight packing of the transcutaneous device 21 in the delivery device 9 is facilitated even if the injection depth varies.

The soft cannula 3 of the transcutaneous device 21 is normally fastened inside the body 1 of the transcutaneous device 21 by an interference fit provided by a bushing 2 inside the body 1, i.e. it is only the friction between cannula 3 and the bushing 2 of the body 1 which keeps the cannula 3 in the correct position. In order to prevent the cannula 3 from sliding back through the upper larger opening in the body 1 of the transcutaneous device 21, the body 1 can be provided with a recess 16 at the proximal surface 17 of the body 1. As the recess 16 creates an open space around the soft cannula 3, it allows the soft cannula 3 to form a small bulge, thereby preventing the soft cannula 3 from sliding back after insertion. This further ensures that the injection depth does not vary and that the delivery device 9 and the transcutaneous device 21 remain fixed in position on the patient's skin and fixed in relation to each other after insertion of the transcutaneous device 21.

### LIST OF REFERENCE NUMBERS

1 Housing/body
1a Top part of two part housing
1b Bottom part of two part housing
1c Upper cylindrical part of the bottom part of the housing
1d Lower cylindrical part of the bottom part of the housing
2 Fastening part/bushing
3 Subcutaneous part/cannula
3a Open end of the subcutaneous part
4 Wall/Membrane
5 Attachment means
5a Upper surface of the attachment means
5b Inclined surface of hooks of prior art
5c Protruding part
6 Piercing member/Insertion needle
7 Receiving portion of base part
7a Protruding parts of receiving portion
7b Contact surface of receiving portion
8 Space around receiving portion
9 Base part/delivery device
9a Receiving portion of the delivery device
10 Adhesive surface of base part/mounting pad
11 Contact area of top surface of sensor part
12 Contact area of subcutaneous surface of sensor part
13 Enhancement view of area mark with circle in FIGS. 4B and 5B
14 Inclining surface on the housing
15 Enhancement view of area mark with circle in FIG. 8A and 9A
16 Recess in the housing
17 Proximal surface of the housing
18 Elastic sealing
19 Outlet opening
20 Opening in the inclining surface
21 Transcutaneous device
22 Top opening

1. A transcutaneous device to be secured to a base part, the base part being secured to a patient's skin during use, the transcutaneous device comprising:
a body;
a subcutaneous part secured unreleasably to the body;
attachment members attaching the transcutaneous device to the base part, the attachment member comprising an area extending an entire length of or part of the length of the body of the transcutaneous device, the area comprises either a continuous layer able to provide a friction extending in the direction of insertion larger than a force attaching the transcutaneous device to an inserting device, or with more than one inwardly or outwardly protruding part sequentially arranged in direction of insertion along an outer surface of the body of the transcutaneous device, the layer or parts provide more than one position in the direction of insertion to attach the transcutaneous device to the base part.

2. The transcutaneous device according to claim 1, wherein the attachment member comprises more than one circumferentially positioned inwardly or outwardly protruding part.

3. The transcutaneous device according to claim 1, wherein the attachment member comprises inwardly or outwardly protruding parts having a length of less than a fourth of a circumference of the body at a level where the part is positioned.

4. The transcutaneous device according to claim 1, wherein at least a part of the outer surface of body of the transcutaneous device is planar and configured to slide and guide the transcutaneous device along an inner surface of an inserter.

5. The transcutaneous device according to claim 1, wherein the attachment member comprises at least three inwardly or outwardly protruding parts sequentially arranged in direction of insertion along the outer surface of the body of the transcutaneous device.

6. The transcutaneous device according to claim 1, wherein the subcutaneous part is a cannula in fluid contact with an internal cavity of the body.

7. The transcutaneous device according to claim 1, wherein the subcutaneous part is a sensor in electric or fluid contact with a part of the body and configured to register one or more components or conditions.

8. The transcutaneous device according to claim 1, wherein the attachment member is cooperating with a corresponding member on the base part.

9. The transcutaneous device according to claim 8, wherein the corresponding member on the base part is flexible to allow passing of one or more protruding parts of the attachment member.

10. The transcutaneous device according to claim 8, wherein the corresponding member comprises a soft and flexible material.

11. The transcutaneous device according to any claim 1, wherein the body comprises a proximal surface through which the subcutaneous part extends.

12. the transcutaneous device according to claim 11, wherein the proximal surface comprises a recess surrounding the subcutaneous part creating an open space around the subcutaneous part.

13. The transcutaneous device according to claim 10 wherein the soft material comprises TPE.

14. The transcutaneous device according to claim 10 wherein the corresponding member is pivotably attached to the base part.
